SEQUENCE LISTING (7 sequences)

SEQ ID NO: 1 - DNA, 590 bp, Oryza sp.
gactgcgtacatgcacggtggctagaaagcactagcattgatcattggagcaacattctgaaggaaaattggaaatcactgcatatcagtagaggcatcatgccttctttgaagcttagctatgatcagctcccttacaatttacaacaaagtttcttgtattgttccatatttcccagtaattatagcttccttagcgaggagttgatcagtatttggatttctcaaggctttgtgcattgtaacgcttcaagtaagagactggaggagatagggagggactacctaactgatttggtgaactctggcttctttcagcaagtttttcgccggggcttctttcagcaagtttttcgccggaacttctttcagcaagttgaacgtacagaatatatcatgtgtggccttatgcatgattttgcaaagttggtttcaaggactgagtacgcaactatagataatgtacagagcaacaaaatactgccaactatacgtcatttgtcaatactaaccgattctgaacactatgaaggtcctagtaacgacaagcttgaagaaagaattagaaatgcagttactcaggactcatc

SEQ ID NO: 2 - DNA, 21 bp, Oryza sp.
gatcattggagcaacattctg

SEQ ID NO: 3 - DNA, 21 bp, Oryza sp.
catttctaattctttcttcaa

SEQ ID NO: 4 - DNA, 17 bp, Oryza sp.
gactgcgtacatgcacg

SEQ ID NO: 5 - DNA, 19 bp, Oryza sp.
gatgagtcctgagtaactg

SEQ ID NO: 6 - DNA, 21 bp, Oryza sp.
ggatgaaatgccaacagcaac

SEQ ID NO: 7 - DNA, 21 bp, Oryza sp.
tcattgcagtggagagctagc

(19) United States
(12) Patent Application Publication
(10) Pub. No.: US 2005/0183173 A1
(43) Pub. Date: Aug. 18, 2005

(54) Fine mapping and application of dna markers linked to a gall midge resistance gene for marker-aided selection in rice

(76) Inventors: Nagesh Sardesai, New Delhi (IN); Arvind Kumar, New Delhi (IN); Suresh Nair, Ali Marg, New Delhi (IN)

Correspondence Address: LADAS & PARRY, 26 WEST 61ST STREET, NEW YORK, NY, 10023, US

(21) Appl. No.: 10/493,001

(22) PCT Filed: Oct. 16, 2002
(86) PCT No.: PCT/IN02/00212

(30) Foreign Application Priority Data
Oct. 17, 2001 (IN) 1065/DEL/2001

(51) Int. Cl. C12Q 1/68; A01H 5/00; C12N 15/82

(52) U.S. Cl. 800/320.2; 435/6; 435/468

(57) ABSTRACT

The present invention relates to fine mapping and potential application of dna markers linked to a gall midge resistance gene gm7 for marker-aided selection in rice. Towards this, the present invention discloses a combination of novel sequence characterized amplified region (SCAR) primers for use in assay with the DNA of Rice plants in question. A cross between the gall midge resistant parent, RP2333 carrying the Gm7 gene and susceptible parent Shyamala, is developed and a F₅ progeny is raised. A polymorphic band is identified from the F₅ progeny, using AFLP that cosegregates with the susceptible phenotype. This band is eluted from the gel and cloned. The cloned AFLP fragment is sequenced and primers are developed for selectively amplifying DNA of susceptible phenotypes, thus differentiating them from the resistant phenotypes. This Gm7 gene linked marker is mapped onto chromosome 4 of rice and is also shown to be linked to Gm2 gene and the blight resistance gene, Xal through fine mapping using Yeast Artificial Chromosomes (YACS) and cosmids. This marker is present in a single copy in the susceptible parent, Shyamala. Primers developed from this marker are able to differentiate between the resistant and susceptible phenotypes in different crosses carrying different gall midge resistance genes. A number of screenings of resistant and susceptible varieties of rice with these primers show consistent polymorphism between them. The use of primers for PCR amplification of DNAs from F₃ progenies derived from crosses between three different parental lines and the primers also differentiates the resistant phenotypes from the susceptible one. The primers of the present invention therefore have a great use in marker assisted selection as they show polymorphism between resistant and susceptible plants and therefore between plants with or without gall midge resistance genes.

### FIELD OF INVENTION

The present invention relates to DNA markers linked to a gall midge resistance gene for marker-aided selection in rice. More particularly, the present invention relates to fine mapping and potential application of DNA markers linked to a gall midge resistance gene, Gm7, for marker-aided selection in rice. The present invention also relates to novel primers for use in preparing the aforesaid DNA markers. The present invention also relates to a method of screening rice varieties for susceptibility and/or resistance to gall midge.

### BACKGROUND OF INVENTION

Rice is the most important crop in the world with over 1.5 billion hectares under paddy cultivation and a worldwide production of over 596 million tons (FAO 1999). Rice crop in the field is subject to attack by a number of insect pests, pathogens, weeds and other harmful organisms. Several studies have reported that major yield losses of rice are often caused by insects alone. Of these insects, a dipteran pest, the rice gall midge (Orseolia oryzae) alone is reported to cause a damage of more than US $ 700 million annually.

Breeding of new rice varieties resistant to gall midge is a traditional and effective method of controlling the damage. However, one of the major problems faced by rice breeders is the emergence of new gall midge biotypes. Different biotypes of gall midge are morphologically indistinguishable and capable of interbreeding but are known to differ in their reaction to genetically defined rice varieties.

Different gall midge resistance genes that provide resistance against different sets of biotypes of the gall midge have been identified and documented (Bentur J. S. and Amudhan S., (1996) ‘Reaction of differentials of different populations of Asian rice gall midge (Orseolia oryzae) under green house condition’, Indian J Agri Sci, 66:197-199). At least six non-allelic gall midge resistance genes in rice (Chaudhary B. P., Shrivastava P. S., Shrivastava M. N., Khush G. S., (1986), ‘Inheritance of resistance to gall midge in some cultivars of rice’, Rice Genetics: Proceedings of the International Rice Genetics Symposium, International Rice Research Institute, The Phillipines, 523-528.) and 5 different biotypes of the gall midge have been identified and reported in India (Behura S. K., Sahu S. C., Rajamani S., Devi A., Mago R, Nair S., and Mohan M, (1999), ‘Differentiation of Asian rice gall midge Orseolia oryzae (Wood-Mason) biotypes by sequence characterised amplified regions (SCARs)’, Insect Mol Biol, 8: 391-397; Sardesai N., Rajyashri K. R., Behura S. K, Nair S., aand Mohan M, (2001), ‘Genetic, physiological and molecular interactions of rice with its major dipteran pest, gall midge’, Plant Cell Tissue Org Cult, 64: 115-131.). Different biotypes of gall midge are distributed in different regions of the country and normally two or more biotypes do not occur together at the same geographical location. Consequently, the selection of rice plants resistant to more than one biotype of gall midge becomes very time consuming since screening is based on the natural occurrence of the pest that is limited to one particular time of the year i.e., just 2-4 months following monsoon. This makes the process of breeding and pyramiding of gall midge resistance genes labor intensive and time consuming. Therefore, there is an urgent need for development of molecular markers that are tightly linked to the gene of interest that would enable one to follow the gene in a cross intended to breed new resistant varieties any time of the year without depending on the annual occurrence of insects (Mohan M., Nair S., Bhagwat A., Krishna T. G., Yano M, Bhatia C. R, and Sasaki T., (1997a), ‘Genome mapping, molecular markers and marker assisted selection in crop plants’ Mol Breed, 3: 87-103.).

### DESCRIPTION OF PRIOR ART

The first gall midge resistance gene to be mapped using restriction fragment length polymorphism (RFLP) and random amplified polymorphic DNA (RAPD) markers was the Gm2 gene (Mohan M., Nair S., Bentur J. S., Prasada Rao U., Bennet J., (1994), ‘RFLP and RAPD mapping of the rice Gm2 gene that confers resistance to biotype 1 of gall midge (Orseolia oryzae)’, Theor Appl Genet, 87: 782-788.) that confers resistance to biotypes 1 and 2 of gall midge (Bentur J. S.; and Amudhan S. (1996), Reaction of differentials of different populations of Asian rice gall midge (Orseolia oryzae) under Green House conditions: Indian J Agric Sci 66:197-199). The present inventors have also tagged and mapped the gall midge resistance gene Gm4t that confers resistance to biotypes 1, 2 and 4 (Nair et al. 1996; Mohan et al. 1997). The potential use of DNA markers linked to the gall midge resistance genes in marker-aided selection (MAS) has also been demonstrated (Nair et al. 1995, 1996).

Biotyping of gall midge is traditionally achieved by observing the infectivity pattern on a set of rice differentials or varieties. It is not possible to morphologically differentiate the different biotypes of gall midge and thus biotyping has been solely based on differential infestation patterns on specific rice hosts. This has the effect of slowing down the process of biotype identification and consequently, the selection for rice plants resistant to more than one biotype of gall midge, particularly since the natural occurrence of gall midge is restricted to a 2 to 4 month period every year. This also slows down the process of breeding new gall midge resistant rice varieties.

### OBJECTS OF THE INVENTION

Accordingly, it is an object of the present invention to provide an alternative to the labour-intensive and time consuming screening procedures of the prior art.

It is another object of the present invention to provide a non destructive method for screening rice populations of interest to select varieties, which are resistant to gall midge attack.

It is another object of the present invention to provide a method for identification of suitable molecular markers closely linked to the gall midge resistance genes to enable easy following of the gene in a cross intended to breed new resistant varieties any time of the year without depending on the annual occurrence of the insects.

It is an important object of the present invention to provide an AFLP marker linked to a gall midge resistance gene for marker-aided selection in rice.

It is still another important object of the present invention to provide a method for fine mapping and potential application of AFLP markers linked to a gall midge resistance gene, Gm7, for marker-aided selection in rice.

It is yet another object of the present invention to provide novel primers for developing AFLP marker linked to a gall midge resistance gene for marker-aided selection in rice.

### SUMMARY OF THE INVENTION

The above and other objects of the present invention are achieved by the tagging of the gene with molecular markers that are closely linked with it, which cosegregate with the desired phenotype. The present invention provides a combination of novel sequence characterized amplified region (SCAR) primers for use in assay with the DNA of the plants in question. A cross between the gall midge resistant parent, RP2333 carrying the Gm7 gene and susceptible parent Shyamala, is developed and a F₅ progeny is raised. A polymorphic band is identified from the F₅ progeny, using AFLP that cosegregates with the susceptible phenotype. This band is eluted from the gel and cloned. The cloned AFLP fragment is sequenced and primers are developed for selectively amplifying DNA of susceptible phenotypes, thus differentiating them from the resistant phenotypes. This Gm7 gene linked marker is mapped onto chromosome 4 of rice and is also shown to be linked to Gm2 gene and the blight resistance gene, Xal, through fine mapping using Yeast Artificial Chromosomes (YACS) and cosmids. This marker is present in a single copy in the susceptible parent, Shyamala. Primers developed from this marker are able to differentiate between the resistant and susceptible phenotypes in different crosses carrying different gall midge resistance genes. A number of screenings of resistant and susceptible varieties of rice with these primers show consistent polymorphism between them. The use of primers for PCR amplification of DNAs from F₃ progenies derived from crosses between three different parental lines and the primers also differentiates the resistant phenotypes from the susceptible one. The primers of the present invention therefore have a great use in marker assisted selection as they show polymorphism between resistant and susceptible plants and therefore between plants with or without gall midge resistance genes.

Thus, according to the present invention, there is provided a combination of sequence characterized amplified region (SCAR) primers for use in marker assisted selection of rice varieties which are resistant to attack by gall midge, said primers having the sequence shown in: Seq ID # 2 and 3 respectively:

| | |
|---|---|
| 5′ - GATCATTGGAGCAACATTCTG - 3′ | Seq ID # 2 |
| and | |
| 5′ - CATTTCTAATTCTTTCTTCAA - 3′ | Seq ID # 3 |

The present invention also provides a method for preparing combination of sequence characterized amplified region (SCAR) primers for use in marker assisted selection of rice varieties which are resistant to attack by gall midge which comprises subjecting genomic DNA extracted from rice varieties resistant to gall midge biotypes and rice varieties susceptible to gall midge biotypes to amplified fragment length polymorphism (AFLP) reactions in the presence of primers, separating the amplified product, extracting DNA therefrom and subjecting it to polymerase chain reaction in the presence of the same primers employed for the AFLP reactions to obtain gall midge susceptible specific AFLP fragment, cloning said AFLP fragment into pGEMT vector to produce a cloned AFLP insert, sequencing said cloned insert and producing said SCAR primers from said clone employing the sequence information, wherein said clone has a nucleotide sequence having Seq. ID. # 1 and said primers have the sequence shown in Seq ID # 2 and 3 respectively:

| | |
|---|---|
| 5′ - GATCATTGGAGCAACATTCTG - 3′ | Seq ID # 2 |
| and | |
| 5′ - CATTTCTAATTCTTTCTTCAA - 3′ | Seq ID # 3 |

Preferably, said rice varieties resistant to gall midge biotypes and rice varieties susceptible to gall midge biotypes from which genomic DNA are extracted are F₅ progenies cross between a rice variety carrying the gene Gm7 conferring resistance to gall midge biotypes 1, 2 and 4 and a rice variety susceptible to gall midge biotypes.

In another preferred embodiment, the cloned AFLP insert comprises susceptibility specific AFLP fragment of 598 bp.

The present method also provides a method for screening a rice variety to determine whether it is resistant or susceptible to gall midge biotypes which comprises extracting DNA from said rice variety, subjecting said rice variety to a polymerase chain reaction amplification in the presence of a combination primers having the sequence shown in Seq ID # 2 and 3 respectively

5′ - GATCATTGGAGCAACATTCTG - 3′
and
5′ - CATTTCTAATTCTTTCTTCAA - 3′

and determining if any fragment of said DNA was amplified, amplification of a fragment indicating the presence of susceptible phenotype specific band thereby indicating that said rice variety is susceptible to Gall midge biotypes and absence of amplification indicating that said rice variety is resistant to Gall midge biotypes.

### DETAILED DESCRIPTION

The present invention will now be described in greater detail with reference to the accompanying drawings in which:

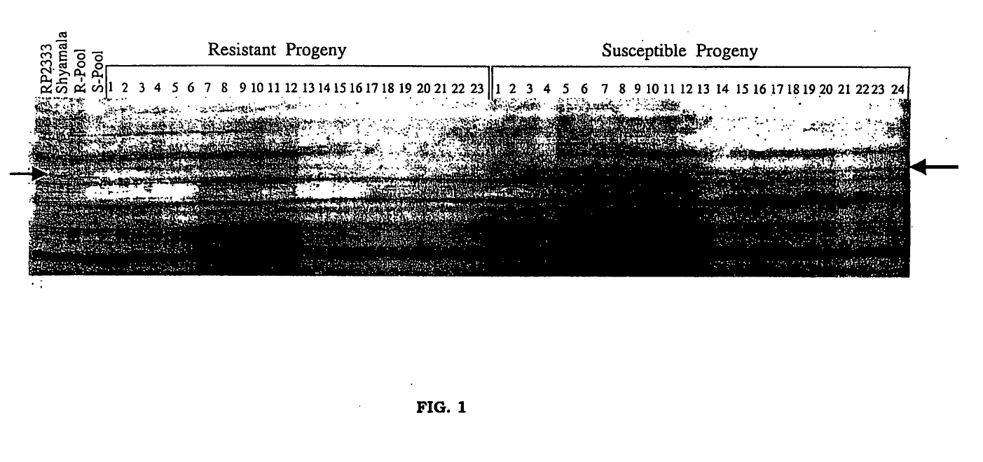

FIG. 1 shows a AFLP fragment segregating with the susceptible phenotypes using primer combination shown in Seq ID # 4 and Seq ID # 5 respectively, i.e., P-CG (5′ GACTGCGTACATGCACG 3′) AND M-CTG (5′ GATGAGTCCTGAGTAACTG 3′).

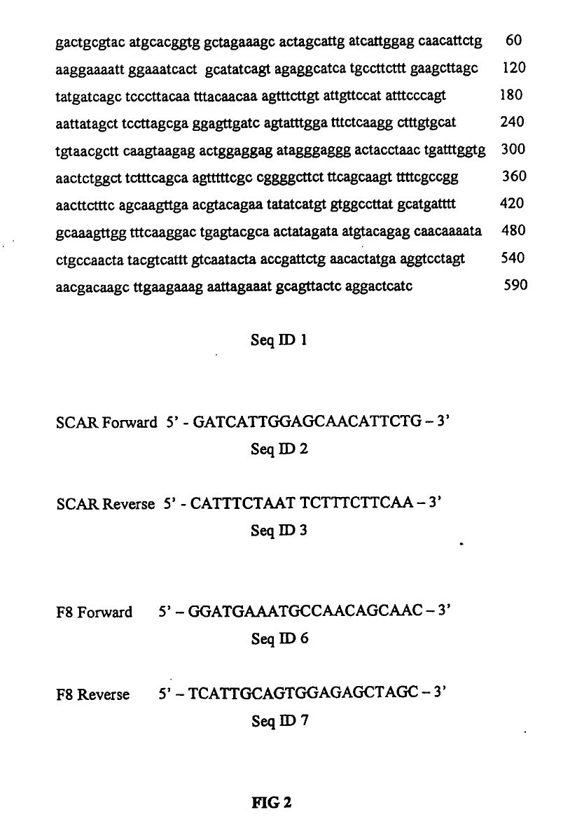

FIG. 2A discloses the complete nucleotide sequence of the susceptible-specific AFLP marker, SA 598, identified in the present invention.

FIG. 2B shows the sequence of SCAR primers (forward and reverse) designed after sequencing the susceptible-specific marker SA598 and sequence of the F8 primers.

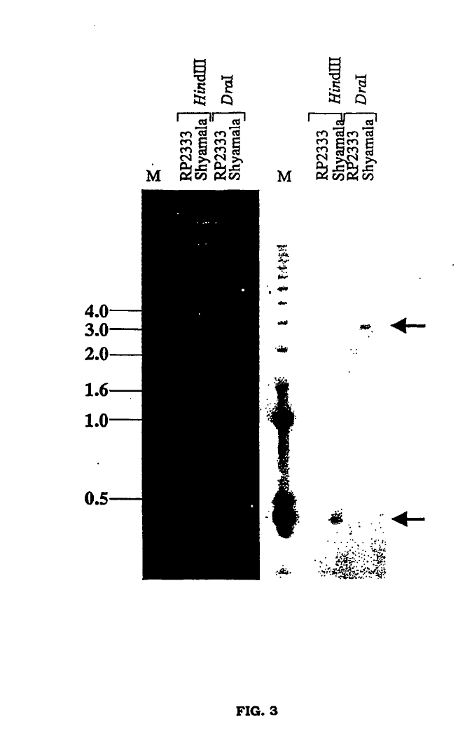

FIG. 3 shows Gel and Southern hybridization of HindIII and DraI digested DNA of RP 2333 and Shyamala probed with SA598.

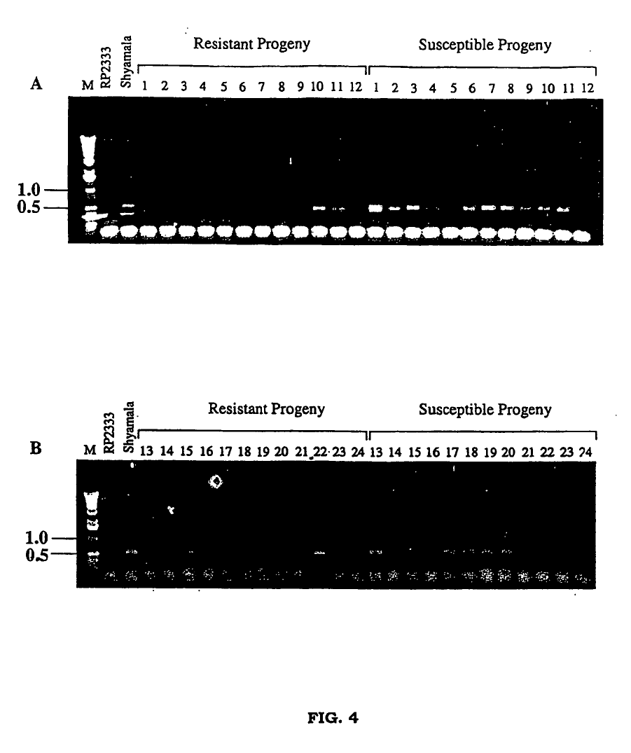

FIG. 4 depicts the PCR-based screening for gall midge-resistant and susceptible progeny in F % population derived from a cross between RP 2333 and Shyamala using the susceptible phenotype-specific SCAR primers.

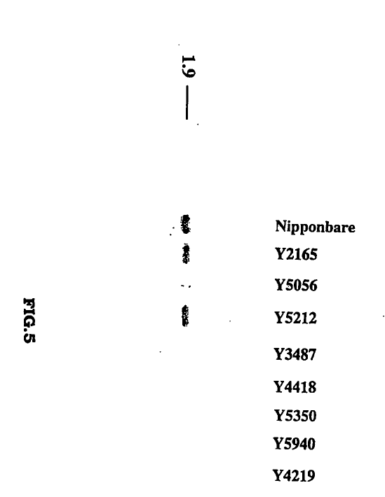

FIG. 5 shows Southern hybridization of DraI digested Nipponbare and YAC DNA, forming a contig encompassing an allele of Gm2 gene with SA 598

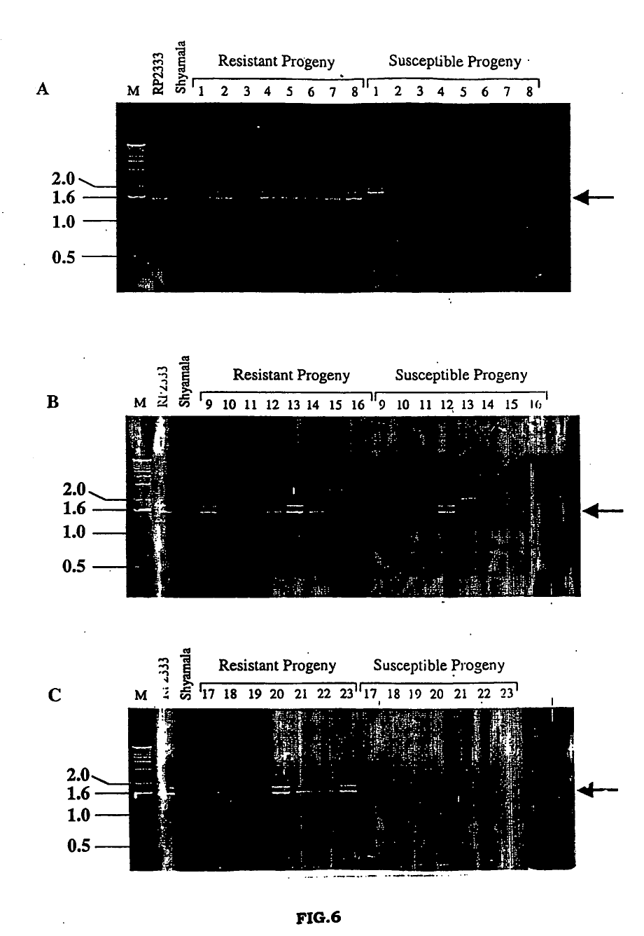

FIG. 6 discloses PCR-based screening for gall midge-resistant and susceptible progeny in F5 population derived from a cross between RP2333 and Shyamala using F8-specific primers.

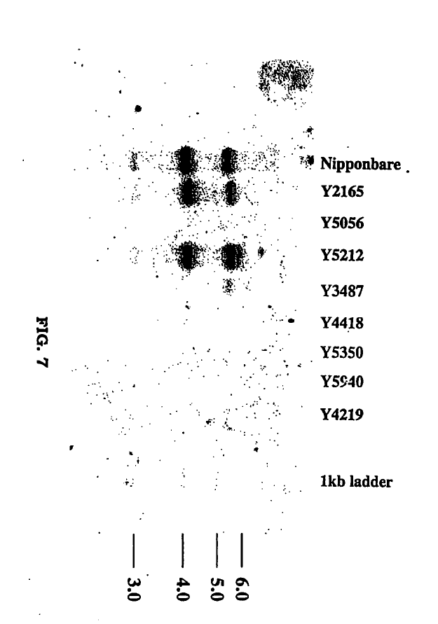

FIG. 7 discloses Southern hybridization of DraI digested Nipponbare and YAC DNAs forming a contig encompaasing an allele of Gm2 gene with F8LB.

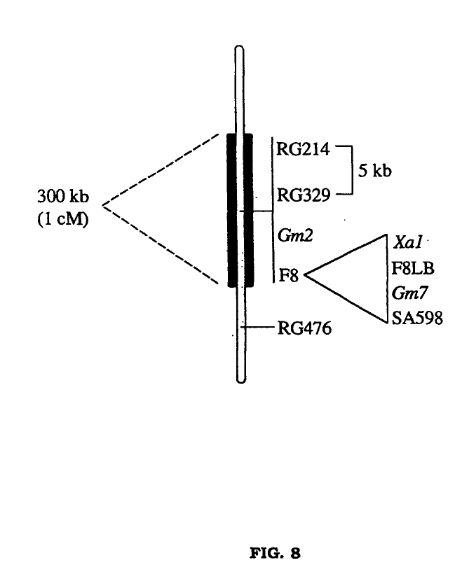

FIG. 8 shows a Graphical representation of a portion of the map of chromosome 4 showing the position of Gm7-linked markers.

The present invention uses bulked segregant analysis (Paran and Michelmore 1993) in the identification of AFLP and RAPD markers linked to a gall midge resistance gene, Gm7, in rice, that have a potential application in marker aided selection. Gm7 has been mapped using a different strategy which involved hybridisation to Bacterial Artificial Chromosomes (BACs), Yeast Artificial Chromosomes (YACs) and cosmids that encompass the Gm2 gene, in the indica rice variety Phalguna. Fine mapping of Gm7 has been reported to reveal a linkage between Gm 7, Gm2 and Xal.

In the present invention, the F₅ population used was derived employing methods known in the art from a cross between the two indica rice varieties, RP 2333 containing Gm 7 (resistant to gall midge biotypes 1, 2 and 4; Kumar et al. 1999) and Shyamla (susceptible to all gall midge biotypes). The scoring for resistance and susceptiblity was done under field conditions at the Indira Gandhi Agricultural University, Raipur, India. The plants were screened for the presence and absence of gall. Plants without any galls were scored as resistant and those having even one gall per plant were scored as susceptible. The scored plants were subsequently harvested for DNA extraction.

RP 2333, carrying the Gm7, gene, was crossed with varieties carrying other gall midge resistance genes to study the allelic relationship between Gm7 and the other gall midge resistance genes. The segregation data of F1, F2 and F3 progenies was recorded.

DNA was isolated from field-grown plants (10 weeks old) using methods known in the art. An equal quantity of DNA from 12 resistant and 12 susceptible F5 individuals was pooled to form the resistant and susceptible bulks, respectively. The concentration of DNA of the two bulks and the two parental DNAs was adjusted to 10 ng/ul. Thereafter, DNA was subjected to Random Amplification.

The amplification conditions employed are well known in the art and will not pose any problem to a skilled artisan. The reaction volume was 25 ul, and 30 ng template DNA was used per reaction. All reactions were carried out on a Perkin-Elmer Cetus DNA Thermal Cycler. Taq DNA polymerase was from Stratagene (La Jolla, Calif.). The RAPD primers used were from the Operon 10-mer Kits (Open Technologies, Alameda, Calif.) 520 random primers of Kits A to Z were utilised in the study. The RAPD product were separated on a 1.1% agarose gel in 1×TBE buffer with 7.5 ul of the 25 ul reaction being loaded on the gel. Gel and buffer contained ethidium bromide at a concentration of 0.5 ug/ml.

The product obtained above was subjected to AFLP reactions (Amplified Fragment Length Polymorphism) by methods known in the art. The method described by (Vos P., Hogers R., Bleeker M, Reijans M., van de Lee T., Hones M, Friyers A., Pot J., Peleman J., Kuiper M., and Zabeau M., (1995), ‘AFLP: a new technique for DNA fingerprinting’, Nucleic Acids Res, 23: 4407-4414) was particularly preferable. Briefly, genomic DNAs (500 ng) from RP2333, Shyamala, the resistant and susceptible bulks and the progenies were digested in a reaction volume of 25 μl. The digested and adapter ligated DNA was amplified with EcoRI or PstI and MseI non-selective primer pairs in a 50 μl reaction. The amplification profile was 94° C. for 30 sec, 56° C. for 30 sec and 72° C. for 1 min for 30 cycles followed by an extension at 72° C. for 5 min. The amplified products were diluted 10-fold in TE (10 mM Tris, 0.5 pH8.0; 1 mM EDTA) and used for selective amplification. EcoRI or PstI primers used in the selective amplification were radiolabeled separately by kinasing 10 ng of each primer with [□-³²P]-ATP. Selective amplification was carried out with 5 μl diluted preamplification product, 1 μl of labeled EcoRI/PstI primer and 50 ng of MseI primer. The reaction volume was 20 μl. A total of 157 primer (MseI and EcoRI/PstI combinations were used in this study. After PCR, 20 ul of formamide dye (containing 98% formamide, 10 mM EDTA, 0.1% bromophenol blue and 0.1% xylene cyanol) was added to the reaction. The samples were heat denatured for 5 min, snap cooled on ice and loaded onto a 6% sequencing gel containing 8M urea. The gel was dried and exposed overnight to X-OMAT-AR film (Kodak) at room temperature.

The DNA was extracted from the gel using methods known in the art. The putatively linked AFLP fragment was first marked out on the dried gel by aligning it with the autoradiogram and cutting out the band along with the gel and soaking the cut piece in an eppendorf containing 100 ul sterile water for 10 min (Behura et al 2000). The gel piece was then boiled in the water for 15 min and the tube spun at 14,000 rpm for 2 min. 10 ul of 3M sodium acetate and 5 ml of glycogen (10 mg/ml) were added to the supernatant. This tube was then spun at 14,000 rpm for 15 min and the pellet washed with 85% ethanol. The pellet was dried and dissolved in 10 ul water.

The eluted AFLP fragment was re-amplified using the same primers and reaction conditions that had revealed the polymorphism and separated on 0.8% agarose. The fragment showing the correct size was excised from the agarose gel and purified using a Qiagen gel extraction kit (Qiagen, Hilder, Germany) for cloning and sequencing of AFLP fragment.

The AFLP fragment considered to be putatively linked to Gm7 was first marked out on the dried gel by aligning it with the autoradiogram and cut out from the gel. The DNA from this gel fragment was isolated as described earlier (Behura S. K, Sahu S. C., Nair S., and Mohan M., (2000), ‘An AFLP marker that differentiates biotypes of the Asian rice gall midge (Orseolia oryzae, Wood-Mason) is sex linked and also linked to avirulence’, Mol Gen Genet, 263:328-334.). The isolated DNA was pelleted, washed with 85% ethanol, dried and dissolved in 10 ul of sterile dH₂O. The DNA (5 ul) was then PCR amplified using the same primer pairs that generated the AFLP fragment. The PCR products were run on a 0.8% agarose gel, gel-purified using a Qiagen gel extraction kit (Qiagen, Hilder, Germany) and cloned into pGEM(T) (Promega, Madison, Wis.). The clone containing the AFLP fragment was named SA598 (susceptibility specific AFLP fragment of 598 bp). DNA sequence of the cloned fragment was determined by the dideoxy chain-termination method using the T7 Sequence Version 2.0 DNA sequencing kit (USB, Cleveland) to develop sequence characterized amplified region (SCAR) primers from the cloned AFLP marker. This cloned AFLP insert was named as SA598 (susceptibility specific AFLP fragment of 598 bp).

Southern hybridisation of AFLP fragment with parental DNA was carried by methods known in the art. Genomic DNA (5 ug) of RP 2333 and Shyamla was digested with 40U each of HindIII and DraI at 37° C. overnight. The digested DNA was run on a 0.8% agarose gel and blotted onto a Hybond nylon membrane as described by [Williams M. N. V., Pande N., Nair S., Mohan M., and Bennet J., (1991)), ‘Restriction fragment length polymorphism analysis of polymerase chain reaction products amplified from mapped loci of rice (Oryzae sativa L.) genomic DNA’, Theor Appl Genet, 82: 489-498.]. SA 598 was excised from the plasmid by restriction with ApaI and NotI and the digested product separated on 0.8% agarose in 1×TBE. The SA598 band was excised from the gel, purified using a Quagen gel extraction kint (Qiagen, Hilder, Germany) and reamplified with the AFLP primers. The membrane was probed with SA 598 labeled with [α³²P]dCTP using the Random Primers DNA Labeling System (Bethesda Research Laboratories, Life Technologies USA.). After hybridisation for 20 h at 650C, the membrane was washed under stringent conditions (twice in 2×SSC at room temperature for 15 min each; once in 2×SSC and 0.1% SDS at 65° C. for 20 min; once in 0.25×SSC and 0.1% SDS at 65° C. for 15 min; and once in 2×SSC at room temperature briefly) and autoradiographed.

Thereafter, a BAC library constructed from the high molecular weight nuclear DNA of the rice variety IR-BB21 (Wang G-L., Holsen T. E., Song W-Y., Wand H-P., and Ronald P. C., (1995), ‘Construction of a rice bacterial artificial chromosome library and identification of clones linked to the Xa-21 disease resistance locus’, Plant J, 7: 525-533.) was probed with [α³²P] dCTP labeled SA 598 as above. The filters were washed after hybridisation at 65° C. for 20 h under stringent conditions (twice in 2×SSC at room temperature for 15 min each; once in 2×SSC and 0.1% SDS at 65° C. for 20 min; once in 0.5×SSC and 0.1% SDS at 65° C. for 20 min; and once in 2×SSC at room temperature briefly) and autoradiographed.

Blots of contiguous stretch of DraI digested YAC DNAs from japonica rice variety, Nipponbare (Rajyashri K. R., Nair S., Ohmido N., Fukui K., Kurata N., Sasaki T, and Mohan M., (1998), ‘Isolation and FISH mapping of yeast artificial chromosomes (YACs) encompassing an allele of the Gm2 gene for gall midge resistance in rice’, Theor Appl Genet, 97:507-514) and of Cosmid DNAs digested with DraI, from indica variety, Phalguna, encompassing the Gm2 gene (data not shown) were hybridised with the SA 598 labeled as above. Filters were washed under stringent conditions as above and autoradiographed.

The forward and reverse primers internal to the 5′ and 3′ end were designed from the sequence of the cloned AFLP fragment (SA 598) using the Oligo 4.0 software (National Biosciences) and were synthesized by Integrated DNA Technology Inc. (USA.) These primers are shown in FIG. 2. These primers were used to amplify genomic DNA from the resistant and susceptible parents as well as resistant and susceptible individuals of the F5 progeny. PCR was carried out in a 50 ul reaction volume containing 10 mM Tris-Cl (pH 8.0) 50 mM KCl, 1.5 mM MgCl2, 0.01% gelatin, 200 uM each dNTP, 380 nM each primer, 125 ng of template DNA and 2.5 U of Taq DNA polymerase. PCR conditions were 94° C. for 1 min, 45° C. for 1 min and 72° C. for 1 min, for 30 cycles.

PCR amplification of parental and bulked DNA with primers specific to Xal gene and RFLP markers (RG329, RG476, RG214, F8, F10) linked to Gm2 gene was also carried out. Genomic DNA of RP2333, Shyamala and the resistant and susceptible bulks was amplified with primers specific to RG329, RG476, RG214 (Yoshimura S., Umehara Y., Kurata N., Nagamura Y., Sasaski T., Minobe Y., and Iwata N., (1996), ‘Identification of a YAC clone carrying the Xa-1 allele, a bacterial blight resistance gene in rice’, Theor Appl Genet, 93:117-122.), F8, F10 (Nair et al. 1995) and primers from the 3′ end of Xal- a bacterial blight resistance gene from rice (Yoshimura et al. 1996; 1998). 200 ng of template DNA was taken for the PCR reaction. PCR conditions were 94° C. for 30 s, 51° C. for 45 s and 72° C. for 1 min; for 30 cycles.

Since only the F8-specific primers (shown in Seq D # 6 and 7) (Nair et al. 1995) showed polymorphism between RP2333 and Shyamala, genomic DNA of the resistant and susceptible individuals of the F₅ population (raised from a cross between RP2333 and Shyamala) was amplified using the same primers. The composition of the reaction mixture and PCR conditions were as described above. The PCR products were electrophoresed on a 1% agarose gel in 1×TBE. The polymorphic band amplified from the resistant parent using the F8 primers was excised from the agarose gel as mentioned above, and isolated using a Qiagen gel extraction kit (Qiagen, Hilder, Germany) and called F8LB (F8 resistance linked band).

Southern hybridization of YACs forming contig encompassing an allele of Gm2 gene with F8LB was carried as follows:

A blot containing YAC DNAs, from clones that encompass an allele of Gm2 gene, digested with DraI (Rajyashri et al. 1998), was hybridized to the gel eluted F8 polymorphic band (F8LB) after labeling with [α³²P]dCTP using the Random Primers DNA Labeling System (Bethesda Research Laboratories, Life Technologies, USA). Hybridisation and washing conditions were same as above.

Cross hybridisation of SA598 with F8LB was achieved by PCR carried out using 100 ng genomic DNA of RP2333, Shyamala and 7 each of the resistant and susceptible individuals of the F₅ population with the SCAR primers and the F8 primers in two separate reactions. PCR conditions were as mentioned for the respective sets of primers. The PCR products were run on a 1% agarose gel in 1×TBE and blotted as described above. Southern hybridization of the blot of F8-primer-amplified PCR products was carried out with SA598 labeled with [α³²P]dCTP using the Random Primers DNA Labeling System (Bethesda Research Laboratories, Life Technologies, USA), whereas, the blot of SCAR-primer-amplified PCR product was hybridized with radiolabeled F8LB. Hybridization was carried out at 65° C. for 20 h and the filters washed under stringent conditions (twice in 2×SSC at room temperature for 20 min each; once in 2×SSC and 0.1% SDS at 65° C. for 20 min; once in 0.1×SSC and 0.1% SDS at 65° C. for 20 min; and once in 2×SSC at room temperature briefly) and autoradiographed.

Genetic analyses of gall midge resistance in RP2333 were carried out. F₂ and F₃ segregation data for resistance to biotype 1 of gall midge in crosses involving the resistant variety RP2333 and susceptible parents, Shyamala and R2270, revealed that the resistance in RP2333 is determined by a single dominant gene (Kumar et al. 1999). Allelic crosses between RP2333 and varieties Samridhi, Phalguna, Abhaya and ARC5984 having the gall midge resistance genes Gm1, Gm2, Gm4 and Gm5, respectively, showed a segregation ratio of 15:1 for resistance: susceptibility in the F₂ and of 7:8:1 for resistance:segregating:susceptible progenies in F₃, indicating the independent segregation of two dominant resistance genes. Crossing between RP2333 with RP2068-18-3-5, the variety harbouring the recessive gm3 gene, showed a segregation ratio of 13:3 for resistance:susceptibility in the F₂ and 7:8:1 in the F₃ progenies (Kumar et al. 1999) indicating the independent segregation of Gm7, the dominant resistance gene and gm3, the recessive resistance gene. This shows that the Gm7 gene is non-allelic to the other gall midge resistance genes reported from India.

The two parental DNAs along with the resistant and susceptible bulks were screened using 520 RAPD primers in order to identify markers linked to Gm7. Of these, 488 primers produced amplification products while the remaining failed to amplify. 24 primers produced resistance-specific products and 8 produced susceptible-specific products in the parents and the bulked DNAs. These primers were further used to screen each of the 12 different individual DNAs that constituted each of the two bulked DNAs. However, none of these primers amplified in a phenotype-specific manner in the individual lines that constituted the bulk thereby indicating that these markers are not closely linked to Gm 7.

In order to identify additional phenotype-specific polymorphisms AFLP was employed. Of the 157 primer combinations used in the present invention, one resistance-specific amplification was identified and 4 showed susceptibility-linked amplification using the EcoRI/MseI combination. Further screening of the resistant and the susceptible individuals of the F₅ progeny (constituting the respective bulked DNAs) using these primer combinations revealed the absence of phenotype-specific amplifications. Using the PstI/MseI primer combinations, 20 combinations identified resistance-specific fragments and 5 combinations showed susceptible-specific amplification in the parents and the bulked DNAs. Analysis of the amplification pattern of the individuals forming the bulks using these primer combinations also showed the absence of phenotype-specific amplification except for the primers combination P-CG×M-CTG which amplified a susceptible-specific fragment in 22 of the 24 susceptible individuals As is clear from FIG. 1, the first two lanes are parents, RP2333 and Shyamala, respectively, followed by the resistant (R-Pool) and susceptible (S-pool) bulks. The remaining lanes are the resistant and susceptible progeny of the F₅ population.

For cloning and Southern Hybridization, the susceptible-specific AFLP fragment (SA598) was eluted from the gel cloned into pGEMT vector and sequenced as shown in FIG. 2. FIG. 2A shows the complete nucleotide sequence of the susceptible-specific AFLP marker, SA598. Sequence information was used to design 21-mer SCAR primers. The sequence of the SCAR primers are shown in FIG. 2B. Southern hybridization of HindIII and DraI digested genomic DNAs of the parents, RP2333 and Shyamala, with SA598 revealed that it is present as a single copy in the susceptible parent. However, the probe did not show any hybridization signal with the genomic DNA of the resistant parent. As can be seen from FIG. 3 the regions of hybridization are indicated by arrows. It can be seen that the probe shows hybridization signals only in Shyamala lanes. The figures on the left shows molecular weight in kb.

As mentioned above, SCAR primers were designed from the sequence information of SA598 and these were used in a PCR assay with the DNA of the parents and resistant and susceptible individuals of the F₅ progeny of the population. To the applicants' knowledge such primers of the present invention as well as their use in nondestructive methods for determining whether or not a rice variety is susceptible to gall midge is not suggested in any prior art. In the present invention, the primers amplified a 0.55 kb fragment in the susceptible individuals. However, it also amplified this fragment in some of the resistant individuals screened as indicated in FIG. 4, which could be because of a rice variety incorrectly scored as a resistant individual due to low insect pressure thereon. In this figure, Panels A and B represent different individuals of the F₅ population. Lane M represents a 1 kb DNA marker ladder. Figures on the left represent molecular weight in kb In order to ascertain the chromosomal location of Gm7, mapping of SA598 was attempted. The IR-BB21 BAC library was screened with this clone. Screening identified BACs that were a subset of the clones that hybridized to YAC probes, Y5212L and F8 (data not included; these markers were earlier shown to flank Gm2 [see Rajyashri et al. 1998]). Southern hybridization of the Nipponbare YAC DNAs with SA598 showed the presence of a single copy of this marker in the japonica variety Nipponbare and the YAC clones, Y2165 and Y5212. The results of the Southern Hybridization are shown in FIG. 5 where the figures on the left represents molecular weight in kb. The insert hybridized to two overlapping cosmids that was previously shown to encompass the Gm2 gene from the indica variety Phalguna (data not shown).

PCR amplification of DNAs from parents and bulks with primers specific to the 3′ end of Xal and RFLP markers linked to Gm2 was carried out. As Gm7 was shown to be linked to F8, a marker previously identified to be linked to Gm2 (Mohan et al. 1994; Rajyashri et al. 1998), it was necessary to determine if any of the other markers linked to Gm2 are linked to Gm7 as well. With the parental and the bulk DNA as templates, primers specific to RG329, RG476, RG214 and Xal amplified fragments of expected size, but the products did not reveal any amplification length polymorphism between the resistant and the susceptible phenotype. The primer set F10 failed to amplify at all. However, the primer set F8 amplified a 1.5 kb fragment that was specific to the resistant phenotype. This can be clearly seen from FIG. 6. Panels A, B and C represent different individuals of the F₅ population. Lane M represents a 1 kb DNA marker ladder. Arrows indicate the polymorphic resistance-phenotype-specific (F8LB) fragment amplified by F8. Figures on the left represent molecular weight in kb. PCR amplification of resistant and susceptible individuals of the F₅ progeny with the F8 set of primers revealed that the 1.5-kb fragment amplified in all the 23 resistant individuals tested except 2, and also amplified in four of the susceptible lines.

Probing the Dra I digested YACs, encompassing an allele of Gm2, with the resistance-linked F8 fragment (F8LB) revealed the presence of 3 bands of 5.5, 4.2 and 3.2 kb in Nipponbare, Y2165 and Y5212 and one band of 5.5 kb in Y3487 as can be seen from FIG. 7. Again, the figures on the right represent molecular weight in kb. Southern hybridizations between the PCR products generated using F8 primer with SA598 as probe and PCR products generated using the novel SCAR primers of the present invention with F8LB as probe failed to reveal any homology between the two markers (data not shown).

Since SA598, hybridizes to the BACs, YACs and cosmids encompassing the Gm2 gene, it can be concluded that this marker is linked to Gm2. Also, as SA598 hybridizes to two of the cosmids to which F8 hybridizes (data not included), it is therefore, logical to conclude that SA598 is linked to Gm7 and is on chromosome 4 and maps along with F8 and Xal (Yoshimura et al. 1996) and F8LB markers as is clear from FIG. 8. In FIG. 8, the darkly shaded bar represents the position of the YAC Y2165. The numbers on the left show genetic (cM) and physical distances in this region of chromosome 4. Gm7-linked markers are on the right of F8 with the genetic (cM) and physical (kb) distances given on the extreme left, within which they are present. The physical and genetic distances are as given earlier.

Genetic analyses of the gall midge resistance gene, Gm7, revealed that it is a dominant gene that is non-allelic to the other known gall midge resistance genes (Kumar et al. 1999). The F₅ population was raised by crossing parents that were different viz-a-viz reaction to different gall midge biotypes. Screening of the parents with over 520 RAPD primers failed to reveal any polymorphism that co-segregated with either the resistance or the susceptibility trait in the individuals of the mapping population. The lack of detectable polymorphisms, that were linked to gall midge resistance, between the two parents could be due to the fact that both parents, RP2333 and Shyamala, are indica varieties. It was therefore, necessary to resort to AFLP, which is known to reveal more polymorphisms in closely related varieties.

AFLP has been used as a DNA fingerprinting technique (Vos et al. 1995) with wide usage in plant genetic studies such as for assessment of genetic diversity in wheat (Barrett B. A. and Kidwell K. K., (1998), ‘AFLP based genetic diversity assessment among wheat cultivars from the Pacific Northwest’, Corp Sci, 38: 1261-1271.), Arabidopsis (Breyne P., Rombaut D., van Gysel A., van Montagu M, Gerats T, (1990), ‘AFLP analysis of genetic diversity within and between Arabidopsis thaliana ecotypes’, Mol Gen Genet, 261: 627-634.) and rice (Zhu J., Gale M. D., Quarrie S., Jackson M. T., and Bryan G. J, (1998), ‘AFLP markers for the study of rice biodiversity’, Theor Appl Genet, 96: 602-611; Aggarwal R. K., Brar D. S., Nandi S., Huang N., and Khush G. S., (1999), ‘Phylogenic relationships among Oryza species revealed by AFLP markers’, Theor Appl Genet, 98: 1320-1328.), for construction of high density genetic maps of barley (Becker J., Vos P., Kuiper M., Salamani F., and Heun M., (1995), ‘Combined mapping of AFLP and RFLP markers in barley’, Mol Gen Genet, 249: 65-73.), maize (Castiglioni P., Ajmone-Marsan P., van Wijk R., and Motto M., (1999), ‘AFLP markers in a molecular linkage map of maize: codominant scoring and linkage group distribution’, Theor Appl Genet, 99: 425-431.) and rice (Mackill D. J., Zhang Z., Redona E. D., and Colowit P. M., (1996), ‘Level of polymorphism and genetic mapping of AFLP markers in rice’, Genome, 39:969-977; Maheswaran M., Subudhi P. K., Nandi S., Xu J. C., Parco A., Yang D. C., and Huang N., (1997), ‘Polymorphism, distribution, and segregation of AFLP markers in a doubled haploid rice population’, Theor Appl Genet, 94:39-45) and for enrichment of DNA markers near a locus of interest in potato (Ballvora A., Hesselbach J., Niewohner J., Leister D., Salamani F., and Gebhardt C., (1995), ‘Marker enrichment and high resolution map of the segment of potato chromosome VII harboring the nematode resistance gene Grol’, Mol Gen Genet, 249:82-90.), Asparagus (Reamon-Büttner S. M., Schodelmaier J., and Jung C., (1998), ‘AFLP markers tightly linked to the sex locus in Asparagus officinalis L.’, Mol Breed, 4:91-98.), tomato (Thomas C. M., Vos P., Zabeau M., Jones D. A., Norcott K. A., Chadwick B. P., and Jones J. D. G., (1995), ‘Identification of amplified restriction fragment polymorphism (AFLP) markers tightly linked to the tomato Cf-9 gene for resistance to Cladosporium fulivum’, Plant J., 8:785-794.), carrot (Bradeen J. M. and Simon P. W., (1998), ‘Conversion of an AFLP fragment linked to the carrot Y₂ locus to a simple codominant PCR based marker form’, Theor Appl Genet, 97:960-967.) and rice (Chen D-H., dela Vina M, Inukai T., Mackill D. J., Ronald P. C., and Nelson R. J., (1999), ‘Molecular mapping of the blast resistance gene Pi44(t), in a line derived from a durably resistant rice cultivar’, Theor Appl Genet, 98:1046-1053.; Xu K., Xu X., Ronald P. C., and Mackill D. J., (2000), ‘A high resolution linkage map of the vicinity of the rice submergence tolerance locus Subl.’, Mol Gen Genet, 263:681-689.). Though, in the present study, screening with over 150 AFLP primer pairs did reveal polymorphisms between the parents, there were very few that were linked to either the resistant or susceptible phenotype in the segregating population. However, AFLP was successful in revealing a 598 base-pair fragment (SA598) that was segregating with the susceptible phenotype in the present study (FIG. 1).

Using the sequence information of the AFLP marker, SA598, primers were synthesized for the sequence characterized amplified region (SCAR) approach. Earlier, RAPD markers have been successfully converted to SCAR markers to make them more robust and reliable (Paran I. and Michelmore R. W., (1993), ‘Development of reliable PCR based markers linked to downy mildew resistance genes in lettuce’, Theor Appl Genet, 85:985-993; Williamson V M, Ho J-Y., Wu F. F., Miller N., and Kaloshian I., (1994), ‘A PCR based marker tightly linked to the nematode resistance gene Mi in tomato’, Theor Appl Genet, 87:757-763; Garcia G. M., Stalker H. T., Shroeder E., Kochert G., (1996), ‘Identification of RAPD, SCAR and RFLP markers tightly linked to nematode resistance genes introgressedfrom Arachis cardenasii into Arachis hypogaea’, Genome, 39:836-845.; Barret et al. 1998; Vidal J. R., Delavault P., Coarer M., Defontaine A., (2000), ‘Design of grapevine (Vitis vinifera L.) cultivar specific SCAR primers for PCR fingerprinting’, Theor Appl Genet, 101:1194-1201.). It may be noted that absence of susceptible phenotype specific band in one of the susceptible individuals of the progeny and the presence of the band in some of the resistant individuals of the progeny may be either due to a recombination event(s) in these phenotypes or because of some escapes as a result of insufficient insect pressure in the case of the individuals scored as resistant.

Earlier, it was difficult to map the Gm4t gene in the population derived from an indica×indica cross (Abhaya×Shyamala) due to lack of polymorphism between them (Mohan et al. 1997b). To overcome this difficulty the marker linked to Gm4t was mapped to chromosome 8 in another mapping population obtained from a japonica×indica cross (Nipponbare×Kasalath) where sufficient polymorphism did exist (Mohan et al. 1997b). Here, faced with a similar problem of lack of polymorphism between the parents, mapping of the Gm7 gene by using YAC, BAC and cosmid clones was attempted which have been previously mapped to chromosome 4.

In this invention, primers specific to F8 (a marker linked to Gm2 [Mohan et al. 1994]) amplified a 1.5 kb fragment (F8LB) in the resistant parent and resistant individuals arising from a cross between RP2333 and Shyamala. This indicates that Gm7 is in the vicinity of Gm2. Further evidence of Gm7 being present on chromosome 4 comes from the results of hybridization of the resistance specific polymorphic band of F8 (F8LB) obtained in this study to YACs that form part of the contig encompassing an allele of Gm2 gene (FIG. 7). These results are in concurrence with the Southern hybridization data of F8 with YAC DNAs where F8 was shown to hybridize to three Dral fragments of 6.0, 4.6 and 3.3 kb in Nipponbare, Y2165 and Y5212, while the 4.6 kb fragment was absent in Y3487 (Rajyashri et al. 1998). Since SA598 hybridized to two cosmid clones, which also hybridize to F8, it was of interest to note that cross hybridisation studies revealed that there was no homology between F8LB and SA598, thus indicating that these markers are distinct and separate physical entities linked to Gm7. It is interesting to note that markers F8, SA598 and F8LB along with the Xal gene, all map to the same cosmids (FIG. 8). Based on the above hybridization data it could be concluded that Gm7 is linked to Gm2 and maps to chromosome 4.

There are various reports that resistance genes to different pests and pathogens such as aphids, nematodes, bacteria, fungi and viruses are linked and located in clusters (Dickinson M. J., Jones D. A., and Jones J. D. G., (1993), ‘Close linkage between the Cf-2/Cf-5 and Mi resistance loci in tomato’, Mol Plant-Microbe Int, 6:341-347; Century K. S., Holub E. B., and Staskawics B. J., (1995), ‘NDR1, a locus of Arabidopsis thaliana that is required for disease resistance to both a bacteria and a fungal pathogen’, Proc Natl Acad Sci USA, 92:6597-6601; Kaloshian L., Lange W. H., and Williamson V. M., (1995), ‘An aphid resistance locus is tightly linked to the nematode resistance gene Mi in tomato’, Proc Natl Acad Sci, USA, 92:622-625; Salmeron J. M., Oldroyd E. D. G., Rommens C. M. T., Scoofield S. R., Kim H-S., Lavelle D. T., Dahlbeck D., and Staskawicz B. J., (1996), ‘Tomato Prf is a member of the leucine rich repeat class of plant disease resistance genes and lies embedded within the Pto kinase gene cluster’, Cell, 86:123-133; Meyers B. C., Chin D. B., Shen K. A., Sivaramakrishnan S., Lavelle D. O., Zhang Z., and Michelmore R. W., (1998), ‘The major resistance gene cluster in lettuce is highly duplicated and spans several megabases’, Plant Cell, 10:1817-1832; Mian M. A. R., Wang T., Phillips D. V., Alvernaz J., and Boerma H. R., (1999), ‘Molecular mapping of the Rcs3 gene for resistance to frogeye leaf spot in soyabean’, Crop Sci, 39:1687-1691; Parniske M. and Jones J. D., (1999), ‘Recombination between diverged clusters of the tomato Cf-9 plant disease resistance gene family’, Proc Natl Acad Sci, USA, 96:5850-5855; Brommonschenkel S. H., Frary A., Frary A., and Tanksley S. D., (2000), ‘The broad spectrum tospovirus resistance gene Sw-5 of tomato is a homolog of the root-knot nematode resistance gene Mi’, Mol Plant-Microbe Int, 13:1130-1138; van der Voort J. R., Kanyuka K., van der Vossen E., Bendahmane A., Mooijman P., Klein-Lankhorst R., Stiekema W., Baulcombe D., and Bakker J., (1999), ‘Tight physical linkage of the nematode resistance gene Gpa2 and the virus resistance gene Rx on a single segment introgressed from the wild species Solanum tuberosum subsp. andigena CPC1673 into cultivated potato’, Mol Plant-Microbe Interact, 12:197-206; van der Vossen E., van der Voort J. N., Kanyuka K., Bendahmane A., Sandbrink H., Baulcombe D. C., Bakker J., Stiekema W., and Klein-Lankhorst R., (2000), ‘Homologues of a single resistance gene cluster in potato confer resistance to distinct pathogens: a virus and a nematode’, Plant J, 23:567-576;). The markers flanking gall midge resistance gene, Gm2, and the bacterial blight resistance gene, Xal, hybridize to the same YAC clone, Y2165 (Rajyashri et al. 1998) to which both, the resistant (F8LB) and the susceptible (SA598) specific markers linked to Gm7 also hybridize, indicating a linkage between Xal and Gm7 gene as well. Thus, linkage between Xal and Gm2 and between Gm2 and Gm7 suggests the presence of Gm7, Gm2 and Xal as a cluster on chromosome 4, in rice.

For marker-based screening to work effectively it is desirable to have all the genes conferring resistance to the various biotypes of gall midge tagged. This would enable the pyramiding of resistance genes against various biotypes into an elite cultivar. Pyramiding of genes is an important strategy in plant breeding for the development of new varieties with durable resistance to several biotypes of an insect pest (Mohan et al. 1997a). The resistance and susceptibility linked markers can be used effectively in a marker aided selection programme for the presence of the Gm7 gene against the gall midge biotypes 1, 2 and 4. With DNA markers also available for two other major gall midge resistance genes (Nair et al. 1995, 1996) there is a potential application in marker-assisted pyramiding of the genes Gm2, Gm4t and Gm7 in rice, as has been reported for blast resistance in rice (Hittalmani et al. 2000) and greenbug resistance in wheat (Porter et al. 2000).

1. A combination of sequence characterized amplified region (SCAR) primers for use in marker assisted selection of rice varieties which are susceptible to attack by gall midge, said primers having the sequence shown in Seq ID Nos. 2 and 3.

2. A method for preparing combination of sequence characterized amplified region (SCAR) primers for use in marker assisted selection of rice varieties which are resistant to attack by gall midge which comprises subjecting genomic DNA extracted from rice varieties resistant to gall midge biotypes and rice varieties susceptible to gall midge biotypes to amplified fragment length polymorphism (AFLP), identifying a polymorphic band using AFLP that cosegregates with the susceptible phenotype, thereby differentiating the susceptible varities from the resistant varieties, eluting the band and cloning it on a vector to obtain a cloned AFLP insert, sequencing said cloned insert and producing said SCAR primers from said clone employing the sequence information, wherein said primers have the sequence shown in Seq IDs Nos. 2 and 3.

3. A method as claimed in claim 2, wherein said AFLP has the nucleotide sequence as shown in Seq. ID. 1.

4. A method as claimed in claim 2, wherein said rice varieties resistant to gall midge biotypes and rice varieties susceptible to gall midge biotypes from which genomic DNA are extracted are Fs progenies of cross between a rice variety carrying the gene Gm7 resistant to gall midge biotypes 1,2 and 4 and a rice variety susceptible to gall midge biotypes.

5. A method as claimed in claim 2, wherein said cloned AFLP insert comprises susceptibility specific AFLP fragment of 598 bp.

6. A method as claimed in claim 2, wherein said vector is a pGEMT vector.

7. A method for preparing combination of sequence characterized amplified region (SCAR) primers for use in marker assisted selection of rice varieties which are resistant to attack by gall midge which comprises subjecting genomic DNA extracted from rice varieties which are Fs progenies of a cross between rice varieties resistant to gall midge biotypes and rice varieties susceptible to gall midge biotypes, to random amplification, extracting the product of random amplification and subjecting it to amplified fragment length polymorphism (AFLP) reactions in the presence of primers, separating the amplified product, extracting DNA therefrom and subjecting it to polymerase chain reaction in the presence of the same primers employed for the AFLP reactions to obtain gall midge susceptible specific AFLP fragment, cloning said AFLP fragment into pGEMT vector to produce a cloned AFLP insert, sequencing said cloned insert and producing said SCAR primers from said clone employing the sequence information, wherein said clone has a nucleotide sequence having Seq. ID. 1 and said primers have the sequence shown in Seq IDs Nos. 2 and 3.

8. A method for screening a rice variety to determine whether it is resistance or susceptible to gall midge biotypes which comprises extracting DNA from said rice variety, subjecting said rice variety to a polymerase chain amplification reaction in the presence of a combination primers having the sequence shown in Seq IDs Nos 2 and 3 and determining if any fragment of said DNA was amplified, amplification of a fragment indicating the presence of susceptible phenotype specific band thereby indicating that said rice variety is susceptible to Gall midge biotypes and absence of amplification indicating that said rice variety is resistant to Gall midge biotypes.

9. A method as claimed in claim 3, wherein said cloned AFLP insert comprises susceptibility specific AFLP fragment of 598 bp.

10. A method as claimed in claim 3, wherein said vector is a pGEMT vector.

11. A method as claimed in claim 4, wherein said vector is a pGEMT vector.

12. A method as claimed in claim 5, wherein said vector is a pGEMT vector.
